(19) United States
(12) Patent Application Publication
(10) Pub. No.: US 2024/0011020 A1
(43) Pub. Date: Jan. 11, 2024

(54) SEQUENCING OLIGONUCLEOTIDES AND METHODS OF USE THEREOF

(71) Applicant: SEQWELL, INC, Beverly, MA (US)

(72) Inventor: Jack T. LEONARD, South Hamilton, MA (US)

(21) Appl. No.: 18/025,343

(22) PCT Filed: Sep. 8, 2021
(86) PCT No.: PCT/US2021/049422

§ 371 Date: Mar. 8, 2023

Related U.S. Application Data
(60) Provisional application No. 63/075,682, filed on Sep. 8, 2020.

(51) Int. Cl. C12N 15/10; C12Q 1/6874

(52) U.S. Cl. CPC C12N 15/1065; C12Q 1/6874

(57) ABSTRACT

Disclosed herein are compositions, kits, and methods for amplifying a sequencing assay region of a target nucleic acid from a nucleic acid sample from any source, while simultaneously adding a plurality of barcode sequences during the amplification process, to create a library of amplified amplicons which is then sequenced, with the barcode sequences enabling identification of the nucleic acid sample from which the amplicon derives. The compositions and methods can be used, for example, to create amplicons containing combinatorial barcodes for the purposes of rapidly sequencing many nucleic acid samples for the presence of viral or mutant nucleic acids.

### BACKGROUND OF THE INVENTION

In nucleic acid assays, the presence of a target nucleic acid sequence can be used for determining the presence or absence of a particular genetic sequence or organisms. Numerous methods exist for identifying the presence of the target nucleic acid sequence. These methods often involve the selective amplification of the target nucleic acid to a quantity above a threshold that then allows the target nucleic acid to be detected. One possible method would be to amplify the target nucleic acid via polymerase chain reaction and then identifying the target via sequencing. However, there are challenges to increasing the multiplexity of such a method to allow simultaneous detection of the target nucleic acid in many samples. Provided herein are compositions and methods for addressing this problem.

### SUMMARY OF THE INVENTION

In general, the present invention relates to oligonucleotides employed in the amplification and barcoding of a target nucleic acid sequence from a nucleic acid sample and methods of use thereof.

In one aspect, the invention provides a pair of sequencing oligonucleotides. The first sequencing oligonucleotide includes, from 5′ to 3′, a first barcode primer region, a first sequencing primer region, a first in-line barcode region, and a first target-specific binding region complementary to a first sequence in a target nucleic acid. The second sequencing oligonucleotide includes, from 5′ to 3′, a second barcode primer region and a second target-specific binding region homologous to a second sequence in the target nucleic acid. The first and second sequences flank a sequencing assay region in the target nucleic acid that can be amplified using the pair.

In some embodiments, the second oligonucleotide further includes a second sequencing primer region between the second barcode primer region and the second target-specific binding region.

In some embodiments, the second oligonucleotide further includes a second in-line barcode region between the second barcode primer region and the second target-specific binding region.

In some embodiments, the sequencing oligonucleotides may include RNA, DNA, or a combination thereof.

In another aspect, the invention provides a kit that includes a pair of sequencing oligonucleotides described herein, as well as a pair of barcoding oligonucleotides. The first barcoding oligonucleotide includes, from 5′ to 3′, a first region for attachment to a solid substrate, a first unique barcode sequence, and a first primer region homologous to the first barcode primer region. The second barcoding oligonucleotide includes, from 5′ to 3′, a second region for attachment to a solid substrate, a second unique barcode sequence, and a second primer region homologous to the second barcode primer region.

In some embodiments, the kit further includes a plurality of pairs of sequencing oligonucleotides, where the sequence of the first in-line barcode region for each first oligonucleotide is different.

In some embodiments the kit further includes a plurality of pairs of barcoding oligonucleotides, where the sequence of the first unique barcode sequence for each first barcoding oligonucleotide is different.

In some embodiments, the kit further includes a plurality of pairs of barcoding oligonucleotides, where the sequence of the second unique barcode sequence for each second barcoding oligonucleotide is different.

In another aspect, the invention provides a method of generating a library from a nucleic acid sample by using a kit described herein to amplify the nucleic acid sample and produce amplicons. The amplicons are nucleic acids that include the first region for attachment to a solid substrate, the first unique barcode sequence, the first barcode primer region, the first sequencing primer region, the first in-line barcode region, the first target-specific binding region, the sequencing assay region, the complement sequence of the second target-specific binding region, the complement sequence of the second barcode primer region, the complement sequence of the second unique barcode sequence, and the complement sequence of the second region for attachment to a solid substrate, and its complementary strand.

In certain embodiments, the method amplifies the nucleic acid sample to produce the library in a single step using the pair of sequencing oligonucleotides and the pair of barcoding oligonucleotides in the same reaction mixture.

In other embodiments, the method amplifies the nucleic acid sample to produce the library in two steps. The first step uses the pair of sequencing oligonucleotides to produce an intermediate amplicon, which is a nucleic acid that includes the first barcode primer region, the first sequencing primer region, the first in-line barcode region, the first target-specific binding region, the sequencing assay region, the complement sequence of the second target-specific binding region, and the complement sequence of the second barcode primer region and its complementary strand. The second step amplifies the intermediate amplicon using the pair of barcoding oligonucleotides to produce the amplicons of the library.

In another aspect, the invention provides a method of sequencing a target nucleic acid sequence in a nucleic acid sample. Provided the amplicons described herein, at least a portion of the amplicons are hybridized to a solid substrate, from which a covalently bound complementary strand is created. The covalently bound complementary strand is then sequenced, which includes sequencing the first in-line barcode region, the first target specific binding region, and the sequencing assay region through sequencing-by-synthesis using a sequencing primer homologous to the first sequencing primer region. The first and second unique barcode sequences of the amplicon are also sequenced.

In some embodiments, the amplicons are hybridized via their first and/or second region for attachment to a solid substrate to immobilized primers covalently attached to the solid substrate.

In some embodiments, the immobilized primer covalently attached to the solid surface is used to generate a complement of the hybridized amplicon through polymerase extension.

In certain embodiments, the first and second unique barcode sequences are sequenced by index reads.

In other embodiments, the first unique barcode is sequenced by index read, and the second unique barcode is sequenced by extending the sequence-by-synthesis step up to the complement sequence of the second unique barcode sequence.

### Definitions

The following definitions are provided for specific terms, which are used in the disclosure of the present invention:

By “amplify” or “amplification” is meant a method to create copies of a nucleic acid molecule. In some instances, the amplification may be achieved using polymerase chain reaction (PCR) or ligase chain reaction (LCR). In other instances, the amplification may be achieved using more than one round of polymerase chain reaction, e.g., two rounds of polymerase chain reaction. In some instances, PCR may be performed using one or more pairs of sequencing oligonucleotides and/or one or more pairs of barcoding oligonucleotides as primers.

By “barcode” is meant a unique oligonucleotide sequence that may allow the corresponding oligonucleotide to be identified. In some embodiments, the nucleic acid sequence may be located at a specific position in a longer nucleic acid sequence. In some embodiments, each barcode may be different from every other barcode by at least a minimum Hamming Distance, wherein the minimum Hamming Distance may be a number greater or equal to 2.

By “complement” or “complementary” sequence is meant the sequence of a first nucleic acid in relation to that of a second nucleic acid, wherein when the first and second nucleic acids are aligned antiparallel (5′ end of the first nucleic acid matched to the 3′ end of the second nucleic acid, and vice versa) to each other, the nucleotide bases at each position in their sequences will have complementary structures following a lock-and-key principle (i.e., A will be paired with U or T and G will be paired with C). Complementary sequences may include mismatches of up to one third of nucleotide bases. For example, two sequences that are nine bases in length may have mismatches of at most 3, at most 2, or at most 1, or at most 0 nucleotide bases, and remain complementary to one another.

By “flank” is meant the relative positions of three nucleic acid regions. A first and second nucleic acid region is said to flank a third nucleic acid region if the first and second regions lie immediately upstream and downstream of the third nucleic acid region.

By “Hamming Distance” is meant a relationship between two nucleic acid sequences of equal length, wherein the number corresponding to the Hamming Distance is the number of bases by which two sequences of equal lengths differ.

By “homologous” is meant having substantially the same sequence. Homologous sequences may differ by up to one third of nucleotide bases. For example, two sequences that are nine bases in length may differ at most by 3, at most by 2, at most by 1, or at most by 0 nucleotide bases, and remain homologous to one another.

By “hybridization” is meant a process in which two single-stranded nucleic acids bind non-covalently by base pairing to form a stable double-stranded nucleic acid. Hybridization may occur for the entire lengths of the two nucleic acids, or only for a portion or subregion of one or both of the nucleic acids. The resulting double-stranded nucleic acid molecule or region is a “duplex.”

By “index read” is meant a method of sequencing a nucleic acid sequence, including a known unique barcode sequence, wherein a sequencing primer is hybridized upstream of the unique barcode sequence, and the nucleic acid read via sequencing-by-synthesis. Index read does not refer to sequencing of the target nucleic acid.

By “library” is meant the amplification product of multiple nucleic acids, wherein the multiple nucleic acids may have the same or different sequences.

By “nucleic acid” is meant a polymeric molecule of at least two linked nucleotides. The terms include, for example, deoxyribonucleic acid (DNA) and ribonucleic acid (RNA), as well as hybrids and mixtures thereof. A nucleic acid may be single-stranded, double-stranded, or contain a mix of regions or portions of both single-stranded or double-stranded sequences. The nucleotides in a nucleic acid are usually linked by phosphodiester bonds, though “nucleic acid” may also refer to other molecular analogs having other types of chemical bonds or backbones, including, but not limited to, phosphoramide, phosphorothioate, phosphorodithioate, O-methyl phosphoramidate, morpholino, locked nucleic acid (LNA), glycerol nucleic acid (GNA), threose nucleic acid (TNA), and peptide nucleic acid (PNA) linkages or backbones. Nucleic acids may contain any combination of deoxyribonucleotides, ribonucleotides, or non-natural analogs thereof. Examples of nucleic acids include, but are not limited to, a gene, a gene fragment, a genomic gap, an exon, an intron, intergenic DNA (including, without limitation, heterochromatic DNA), messenger RNA (mRNA), transfer RNA, ribosomal RNA, ribozymes, small interfering RNA (siRNA), miRNA, small nucleolar RNA (snoRNA), cDNA, recombinant polynucleotides, branched polynucleotides, plasmids, vectors, isolated DNA of a sequence, isolated RNA of a sequence, nucleic acid probes, and primers.

By “nucleotide” is meant any deoxyribonucleotide, ribonucleotide, non-standard nucleotide, modified nucleotide, or nucleotide analog. Nucleotides include adenine, thymine, cytosine, guanine, and uracil. Examples of modified nucleotides include, but are not limited to, diaminopurine, 5-fluorouracil, 5-bromouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil.

By “oligonucleotide” is meant a nucleic acid up to 150 nucleotides in length. Oligonucleotides may be synthetic. Oligonucleotides may contain one or more chemical modifications, whether on the 5′ end, the 3′ end, or internally. Examples of chemical modifications include, but are not limited to, addition of functional groups (e.g., biotins, amino modifiers, alkynes, thiol modifiers, or azides), fluorophores (e.g. quantum dots or organic dyes), spacers (e.g. C3 spacer, dSpacer, photo-cleavable spacers), modified bases, or modified backbones.

By “sequencing-by-ligation” is meant a method of sequencing a nucleic acid, wherein multiple cycles of ligation sequencing are performed. In each cycle of ligation sequencing, a ligation primer is first hybridized immediately upstream of the region of a target nucleic acid to be sequenced, and multiple rounds of ligation are performed. In each round of ligation, a pool of short oligonucleotides (typically containing 8 or 9 nucleotides but can be shorter or longer) is presented to the nucleic acid being sequenced, and the best matching complementary sequence will be ligated. The identity of one or more nucleotides on the short oligonucleotides is typically encoded via a fluorophore, wherein imaging following each round of ligation can determine the identity of the bases on the nucleic acid being sequenced in the corresponding positions. Multiple rounds of ligation are performed until the end of the nucleic acid being sequenced. The ligated strand can then be removed, and a new ligation primer one or more bases away from the previous ligation primer can be used to begin a new cycle of ligation sequencing. Multiple cycles of ligation sequencing are performed until the identity of the entire nucleic acid being sequenced has been determined.

By “sequencing-by-synthesis” is meant a method of sequencing a nucleic acid, wherein a sequencing primer is first hybridized immediately upstream of the region of a target nucleic acid to be sequenced, and multiple rounds of sequencing cycles are performed. During each sequencing cycle, a single, complementary, detectable, e.g., fluorescently labeled, nucleotide is added to the nucleic acid downstream of the extending sequencing primer. The sequence of the target nucleic acid is then determined based upon the fluorescent signals observed during each sequencing cycle. It will be understood that the sequence of a sequencing assay region can be determined by sequencing the sense or antisense strand or both.

By “sequence in-line” is meant a method of sequencing a nucleic acid sequence, wherein the nucleic acid sequence is sequenced by extending a sequencing-by-synthesis reaction to include one or more nucleic acid sequences that lie downstream of the same strand of nucleic acid undergoing sequencing-by-synthesis.

By “target nucleic acid” is meant any nucleic acid (e.g., RNA or DNA) of interest that is selected for amplification or analysis (e.g., sequencing) using a composition (e.g., sequencing oligonucleotides or barcoding oligonucleotides) or method of the invention. In some instances, RNA may be converted to cDNA prior to being treated with a composition of the invention (e.g., sequencing oligonucleotides or barcoding oligonucleotides).

### BRIEF DESCRIPTION OF THE DRAWINGS

FIG. 1. Two versions of sequencing oligonucleotide pairs, (A/B) and (A/B2). A first sequencing oligonucleotide (A) having a first barcode primer region (1) complementary to a first barcoding primer, a first sequencing primer region (2), a first in-line barcoding region (3), and a first target-specific binding region (4). A second sequencing oligonucleotide (B) having a second barcode primer region (5) complementary to a second barcoding primer, an optional second sequencing primer region (6); and a second target-specific binding region (7). An alternative second sequencing oligonucleotide (B2) having a second barcode primer region (5) complementary to a second barcoding primer, and a second target-specific binding region (7).

FIG. 2. A first sequencing oligonucleotide (A) having a first barcode primer region (1), a first sequencing primer region (2), a first in-line barcoding region (3); and a first target-specific binding region (4). A target nucleic acid (8), having a region (4′) that is complementary to and hybridized with (4).

FIG. 3. A first sequencing oligonucleotide (A) having a first barcode primer region (1), a first sequencing primer region (2), a first in-line barcoding region (3), and a first target-specific binding region (4). A target nucleic acid (8) having a region (4′) that is complementary to and hybridized with (4), a sequencing assay region (c), and a second target-specific binding region (7). A first polymerase extension (8′) using the target nucleic acid (8) as template for synthesis, having extended regions (c′) and (7′) that are the reverse complements of the sequencing assay region (c) and of the second target-specific binding region (7), respectively. A second sequencing oligonucleotide (B) having a second barcode primer region (5), an optional second sequencing primer region (6), and a second target-specific binding region (7) complementary to and hybridized with region (7′) on the polymerase extension (8′).

FIG. 4. A first sequencing oligonucleotide (A) having a first barcode primer region (1), a first sequencing primer region (2), a first in-line barcoding region (3); and a first target-specific binding region (4). A target nucleic acid (8) having a region (4′) that is complementary to and hybridized with (4), a sequencing assay region (c), and a second target-specific binding region (7). A first polymerase extension (8′) using the target nucleic acid (8) as template for synthesis, having extended regions (c′) and (7′) that are the reverse complements of the sequencing assay region (c) and of the second target-specific binding region (7), respectively. A second sequencing oligonucleotide (B) having a second barcode primer region (5), an optional second sequencing primer region (6), and a second target-specific binding region (7) complementary to and hybridized with region (7′) on the polymerase extension product (8′). A second polymerase extension product (10) using the first polymerase extension product (8′) as template for synthesis. The second polymerase extension product (10) having a sequencing assay region (c) and regions (4′), (3′), (2′) and (1′), which are the reverse complements of regions (c′), (4), (3), (2) and (1) on the first sequencing oligonucleotide (A), respectively.

FIG. 5. An amplified intermediate amplicon (11) having regions (1′) and (5′) homologous to the first and second barcoding primers, respectively; first and second sequencing primer regions (2) and (6); a first in-line barcode region (3); target-specific primer regions (4) and (7); a sequencing assay region (c); and regions (2′), (3′), (4′), (6′), (7′) and (c′) that are the reverse complements of regions (2), (3), (4), (6), (7) and (c), respectively.

FIG. 6. Opposite strands of an intermediate amplicon hybridized to a first (X) and second (Y) barcoding oligonucleotide. A first barcoding oligonucleotide (X) having a first region for attachment to a solid substrate (13), a first unique barcode sequence (12), and a first primer region (1″) that is homologous to the first barcode primer region (1). A second barcoding oligonucleotide (Y) having a second region for attachment to a solid substrate (15), a second unique barcode sequence (14), and a second primer region (5″) that is homologous to the second barcode primer region (5).

FIG. 7. Polymerase extensions of the first (X) and second (Y) barcoding oligonucleotides using opposite strands of the intermediate amplicon as template for synthesis. The extension products of a second barcoding oligonucleotide (Y) having regions (15), (14), (5″), (6), (7), (c), (4′), (3′), (2′), and (1′); and their respective complements on the opposite strand. The extension products of a first barcoding oligonucleotide (X) having regions (13), (12), (1″), (2), (3), (4), (c′), (7′), (6′), (5′), and their respective complements on the opposite strand. The 3′-terminal portions of all four polymerase extension products are of particular importance because they serve as the priming sites for the barcoding oligonucleotides in subsequent rounds of amplification.

FIG. 8. An amplicon in an amplified library (16) after multiple rounds of amplification with first (A) and second (B) sequencing oligonucleotides and first (X) and second (Y) barcoding oligonucleotides. Vertical dotted lines in the figure show the positions of the 3′-termini of the sequencing and barcoding oligonucleotides relative to the corresponding positions in the amplicon (16). The amplicon (16) having a first region for attachment to a solid substrate (13), a first unique barcode sequence (12), a first barcode primer region (1), a first sequencing primer region (2), a first in-line barcode region (3), a first target-specific binding region (4), a sequencing assay region (c), a second target-specific primer region (7), an optional second sequencing primer region (6), a second barcode primer region (5), a second unique barcode sequence (14), and a second region for attachment to a solid substrate (15), as well as complementary sequences (13′), (12′), (1′), (2′), (3′), (4′), (c′), (7′), (6′), (5′), (14′), and (15′). A first barcoding oligonucleotide (X) having a first region for attachment to a solid substrate (13), a first unique barcode sequence (12), and a first primer region (1″) that is homologous to the first barcode primer region (1). A second barcoding oligonucleotide (Y) having a second region for attachment to a solid substrate (15), a second unique barcode sequence (14), and a second primer region (5″) that is homologous to the second barcode primer region (5). A first sequencing oligonucleotide (A) having a first barcode primer region (1), a sequencing primer region (2), a first in-line barcoding region (3), and a first target-specific binding region (4). A second sequencing oligonucleotide (B) having a second barcode primer region (5), an optional second sequencing primer region (6); and a second target-specific binding region (7).

FIG. 9. An immobilized primer (17) covalently attached to a solid surface for sequencing (18). A single-stranded, amplicon (19) hybridized to the complementary immobilized primer (17). A polymerase extension (20) of the immobilized primer (17) using the single-stranded amplicon (19) as template.

FIG. 10. After clonal amplification and denaturation, a single-stranded amplicon (21) remains covalently attached to the solid surface for sequencing (18). A sequencing primer (2″) is hybridized to the complementary region in the single-stranded amplicon (15). Sequencing-by-synthesis initiates at the 3′-terminus of sequencing primer (2″) using the immobilized library fragment (21) as template. The sequencing extends through a first in-line barcode sequence region (3); a target-specific primer region (4); and a complementary sequence to the sequencing assay region (c′). The unique barcode sequences or complements thereof (12 and 14′) are sequenced in separate index reads.

FIG. 11. After clonal amplification and denaturation, a single-stranded library fragment (22) remains covalently attached to the solid surface for sequencing (18). A sequencing primer (2″) is hybridized to the complementary single-stranded library fragment (15). Sequencing-by-synthesis initiates at the 3′-terminus of the sequencing primer (2″) using the immobilized library fragment (22) as template. The sequencing extends through a first in-line barcode sequence region (3); a target-specific binding region (4); a complementary sequence to the sequencing assay region (c′), a second target-specific primer region (7′), and a complementary sequence of the second unique barcode sequence (14′). The complementary sequence of the first unique barcode sequence (12′) is sequenced in a separate index read.

FIG. 12. A representation of the sequencing results from the experiment described in Example 1. For reactions receiving different amounts of target (SARS-CoV-2 copies), the number of reads mapping to N1 and N2 were normalized by dividing by the total number of reads mapping to RP (internal control).

FIG. 13. An illustration how 384 unique pairs of first (X) and second (Y) barcoding oligonucleotides can be reused to generate 2,304 barcode combinations (Z) with the addition of a limited number (n=6) of pairs of sequencing oligonucleotides (A) and (B) in which the in-line barcode sequence is different for each of the first sequencing oligonucleotides (A1-A6).

FIG. 14. The number of new oligonucleotides required to increase the number of barcode combinations upward from 384 as described in Example 2. The data points represented by black circles (New_seq_oligos) show the number of new sequencing oligonucleotides needed to increase barcode combinations, while the data points represented by white triangles (UDI_bc_oligos) show the number of new barcode oligonucleotides that would be required if sequencing oligonucleotides were not used to increase barcode combinations.

### DETAILED DESCRIPTION

We have developed new oligonucleotides and methods of their use that increase the number of samples that can be sequenced in parallel. Disclosed herein are compositions, kits, and methods for amplifying a sequencing assay region of a target nucleic acid from a nucleic acid sample from any source, while simultaneously adding a plurality of barcode sequences during the amplification process, to create a library of amplified amplicons which is then sequenced, with the barcode sequences enabling identification of the nucleic acid sample from which the amplicon derives. The compositions and methods herein can be used in a variety of applications, particularly those identifying the sequence of a target nucleic acid from nucleic acid samples in a highly multiplexed manner. The inventive approach combines the high specificity and sensitivity of qPCR assays with the high detection resolution and throughput offered by next-generation sequencing (NGS) methods by leveraging PCR amplification to encode NGS reads with additional barcoding regions in a combinatorial manner. The compositions and methods can be used, for example, to create amplicons containing combinatorial barcodes for the purposes of rapidly sequencing many nucleic acid samples for the presence of viral or mutant nucleic acids.

NGS is a powerful tool in molecular biology. The technology involves millions of nucleic acid strands being read in parallel, one base at a time. Depending on the method used, the DNA strand is read from one or both ends of the DNA molecule. To leverage the growing raw sequencing output of NextGen Sequencing platforms for more samples, barcode sequences (indexes) were incorporated by manufacturers into the synthetic adapters used for NGS library construction. Later during data analysis, the barcode sequences were used to assign sequencing reads to specific samples. Using conventional library preparation methods, barcode sequences could either be encoded in the adapter at one end (single-index sequencing) or in the adapters at both ends (dual-index sequencing).

Over the past decade, DNA sequencing systems have evolved from a throughput of several megabases per day to a throughput of terabases per day, including the use of patterned flow cells that provide known locations and dimensions. This increase in throughput has provided the capacity to simultaneously sequence DNA from multiple sources of nucleic acids using multiplexed libraries. Despite the improvements to throughput, however, the scientific community has reported instances of the misassignment of reads in multiplex libraries, coming from a switch to a new exclusion amplification (ExAmp) technology.

Unique dual index (UDI) sequencing is the current industry standard for DNA sequencing because UDIs address the challenges of crosstalk and read contamination between samples, which lead to sample misassignment. When preparing samples for sequencing on the Illumina® sequencing systems, unique dual indexes (i5 and i7 barcodes) are added to the 5′ and 3′ ends of NGS library fragments during library amplification with primers carrying unique pairs of barcodes or by ligation of adapters carrying unique pairs of barcodes.

The advantage of labeling samples using UDIs is realized when libraries derived from separate samples are sequenced together on the same run and analyzed. Reads carrying the expected barcode combination can be distinguished from reads carrying unexpected barcode combinations arising from cross-contamination of reagents, misincorporation of barcode sequences during amplification on the sequencing system, or optical crosstalk during data acquisition. Reads carrying the expected barcode combinations are computationally assigned to each corresponding sample, while reads carrying unexpected barcode combinations are discarded (i.e., are not used for analysis).

Modern NGS systems typically generate millions of paired reads per sequencing run. For example, Illumina sequencing systems generate as few as 1 million paired reads per run for small desktop sequencers such as the MiSeq™ System, and up to 10 billion paired reads per run for large production scale sequencers such as the NovaSeg™ 6000 System.

Small nucleic acid targets, such as 300 bp amplicons, rarely require a depth of sequencing greater than 100× to confidently determine the DNA sequence. If 100× was set as the minimum threshold for coverage, a paired read configuration of 2×151 bases could be applied to sequence a 300 bp amplicon. If amplicons were then prepared from 384 samples and UDIs were added to uniquely label library fragments from each sample, those 384 samples could be analyzed in a single NovaSeq™ 6000 sequencing run. If 10 billion read pairs were obtained, the average number of UDI read pairs per sample would be approximately 26 million (10 billion read pairs/384 samples). In this example, 26 million read pairs would be an extremely unproductive use of sequencing output because the minimum threshold for sequencing depth is 100×, i.e., only requiring 100 read pairs per sample. This example illustrates that many more samples could be sequenced per run if more than 384 UDIs were readily available. However, most commercially available UDIs are available as a maximum of 384 pairs of UDIs. The number of UDIs needed to scale-up the number of samples per sequencing run increases in a linear fashion. Currently, to achieve higher levels of multiplexing with UDIs, larger sets of UDI-primers or UDI-adapters would need to be validated, manufactured, and quality-controlled before use.

### Compositions

The compositions of the invention include a pair of sequencing oligonucleotides that allow the insertion of an in-line barcode in the resulting nucleic acid product of an amplification reaction. The sequencing oligonucleotides may be employed with a pair of barcoding oligonucleotides that allow the insertion of an additional pair of unique barcode sequences, e.g., UDIs, to the nucleic acid product of the amplification reaction.

### Sequencing Oligonucleotides

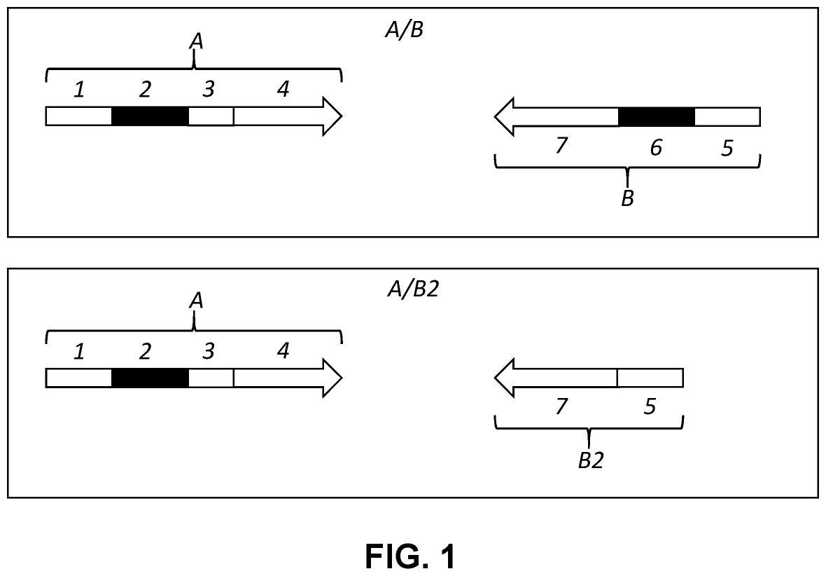

The invention provides compositions that include a pair of sequencing oligonucleotides. As depicted in FIG. 1, a pair of sequencing oligonucleotides includes a first oligonucleotide having, from 5′ to 3′, a first barcode primer region, a first sequencing primer region, a first in-line barcode region, and a first target-specific binding region complementary to a first sequence in a target nucleic acid; and a second oligonucleotide having, from 5′ to 3′, a second barcode primer region and a second target-specific binding region homologous to a second sequence in the target nucleic acid. As further depicted in FIG. 1, the second oligonucleotide may further include a second sequencing primer region between the second barcode primer region and the second target-specific binding region, which can permit sequencing in the opposite direction as compared to the first sequencing primer region. In some embodiments, the second oligonucleotide may further contain a second in-line barcode region between the second barcode primer region and the second target-specific binding region to allow for further combinatorial barcoding.

Each region of the sequencing oligonucleotide may include 5-30 nucleotides. For example, the barcode primer regions may include 7-20 nt; the sequencing primer regions may include 12-30 nt; the in-line barcode regions may include 5-18 nt; and the target-specific binding region may include 5-30 nt. The overall sequence of the oligonucleotides is chosen to be non-naturally occurring. In certain embodiments, the in-line barcode regions are immediately 3′ of the barcode primer region, allowing for determination of the in-line barcode sequence first. In some embodiments, the sequencing oligonucleotides may include RNA, DNA, or a combination thereof. The sequencing oligonucleotides may also contain modified nucleotides, e.g., modified bases, sugars, or phosphates. In one embodiment, uracil is substituted for positions where thymine appears in the sequencing oligonucleotides, which allows removal of trace amounts of synthetic oligonucleotide and carryover PCR products by pretreatment with uracil-DNA glycosylase (UDG).

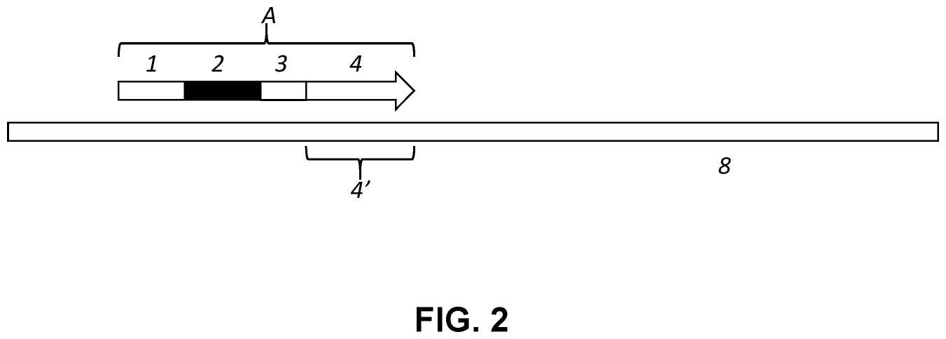

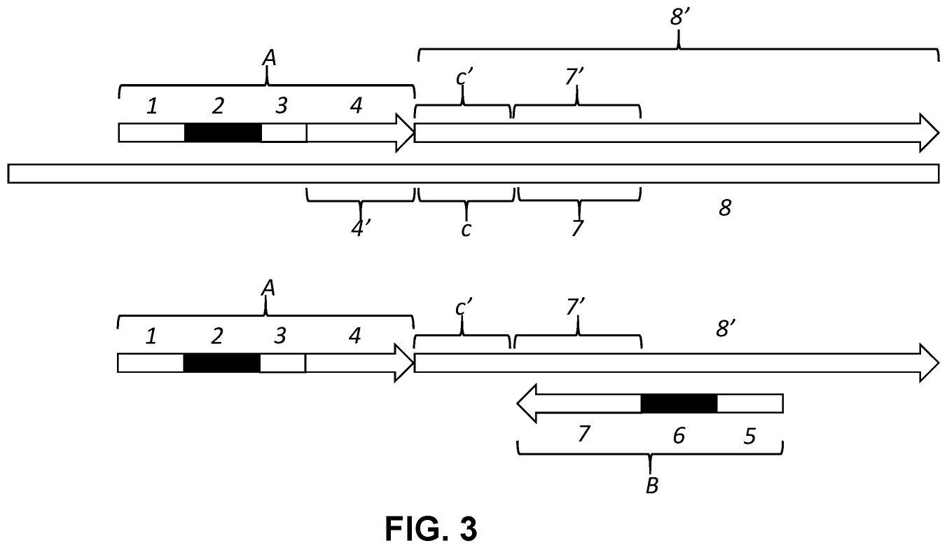

The first and second target-specific binding regions flank a sequencing assay region in the target nucleic acid and allow for amplification thereof. As depicted in FIG. 2 and FIG. 3, the pair of sequencing oligonucleotides can be used as primers in a nucleic acid amplification reaction of the target nucleic acid by hybridizing via the first and second target-specific binding regions, which bind to opposite strands in amplification.

In certain embodiments, the pair of sequencing oligonucleotides may not contain a first or second target-specific binding region. Instead, the first sequencing oligonucleotide would include, from 5′ to 3′, a first barcode primer region, a first sequencing primer region, and a first in-line barcode region. The second sequencing oligonucleotide could either include only a complementary sequence of a second barcode primer region; from 5′ to 3′, a complementary sequence of a second barcode primer region and a complementary sequence of a second sequencing primer region; or, from 5′ to 3′, a complementary sequence of a second barcode primer region, a complementary sequence of a second sequencing primer region, and a complementary sequence of a second in-line barcode region. In other embodiments, the first sequencing oligonucleotide would include, from 5′ to 3′, a complementary sequence of a first barcode primer region, a complementary sequence of a first sequencing primer region, and a complementary sequence of a first in-line barcode region. The second sequencing oligonucleotide could either include only a second barcode primer region; from 5′ to 3′, a second barcode primer region and a second sequencing primer region; or, from 5′ to 3′, a second barcode primer region, a second sequencing primer region, and a second in-line barcode region. In some embodiments, the sequencing oligonucleotides may include RNA, DNA, or a combination thereof.

### Barcoding Oligonucleotides

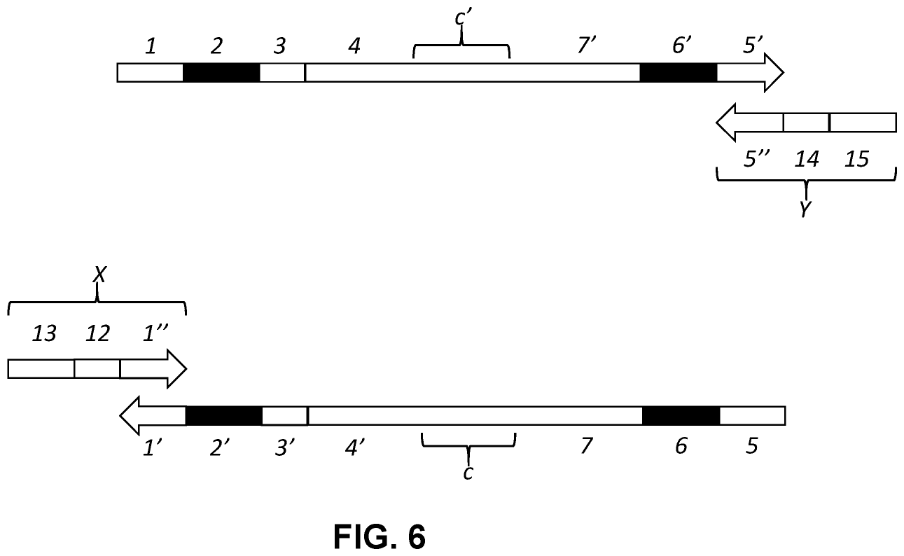

The invention further provides compositions that include a pair of barcoding oligonucleotides. As depicted in FIG. 6, a pair of barcoding oligonucleotides includes a first oligonucleotide including, from 5′ to 3′, a first region for attachment to a solid substrate, a first unique barcode sequence, and a first primer region homologous to the first barcode primer region; and a second oligonucleotide including, from 5′ to 3′, a second region for attachment to a solid substrate, a second unique barcode sequence, and a second primer region homologous to the second barcode primer region.

Each region of the barcoding oligonucleotide may include 5-20 nucleotides. For example, the unique barcode sequences may have 5-18 nt and the primer regions may have 7-20 nt. The regions for attachment to a solid substrate, e.g., P5 and/or P7, may have 12-30 nt. The overall sequence of the oligonucleotides is chosen to be non-naturally occurring. In certain embodiments, the unique barcode sequences are a UDI pair. In some embodiments, the barcoding oligonucleotides may include RNA, DNA, or a combination thereof. The barcoding oligonucleotides may also contain modified nucleotides, e.g., modified bases, sugars, or phosphates. In one embodiment, uracil is substituted for positions where thymine appears in the barcoding oligonucleotides, which allows removal of trace amounts of synthetic oligonucleotide and carryover PCR products by pretreatment with uracil-DNA glycosylase (UDG).

As further depicted in FIG. 6, the pair of barcoding oligonucleotides can be used as primers in an amplification reaction in conjunction with a pair of sequencing oligonucleotides and a target nucleic acid sequence, wherein the first and second barcode primer region sequences hybridize to their complement sequences during the amplification reaction.

### Kits

The invention provides kits and other combinations of the oligonucleotides. For example, a kit may include a plurality of pairs of sequencing oligonucleotides, where each pair of sequencing oligonucleotides may have different in-line barcodes and optionally are otherwise the same. For example, a kit may include 2, 3, 4, 5, 6, 7, 8, 10, 12, 14, 16, 18, 20, 22, 24, 32, 64, 96, 100, 128, 150, 200, 250, 256, 300, 350, 384, 400, 500, 512, 600, 700, 800, 900, 1000, or more pairs of sequencing oligonucleotides with different in-line barcode regions. A kit may also include a plurality of pairs of barcoding oligonucleotides, where the sequence of the first unique barcode sequence for each first barcoding oligonucleotide is different and optionally the remaining sequences are identical. For example, a kit may include 2, 3, 4, 5, 6, 7, 8, 10, 12, 14, 16, 18, 20, 22, 24, 32, 64, 96, 100, 128, 150, 200, 250, 256, 300, 350, 384, 400, 500, 512, 600, 700, 800, 900, 1000, or more first barcoding oligonucleotides, where the first unique barcode sequences are different. In some embodiments, the pairs of barcoding oligonucleotides include a second unique barcode sequence, where each second barcoding oligonucleotide is different. For example, a kit may include 2, 3, 4, 5, 6, 7, 8, 12, 14, 16, 18, 20, 22, 24, 32, 64, 96, 100, 128, 150, 200, 250, 256, 300, 350, 384, 400, 500, 512, 600, 700, 800, 900, 1000, or more second barcoding oligonucleotides, where the second unique barcode sequences are different and optionally the remaining sequences are identical. Two different pairs of barcoding oligonucleotides are considered different whether they differ by only their first barcoding oligonucleotides, by only their second barcoding oligonucleotides, or by both their first and second barcoding oligonucleotides.

For a given amplification reaction, the barcode primer regions of the sequencing oligonucleotides and the primer regions of the barcoding oligonucleotides are homologous. In certain embodiments, the sequences are identical. In certain embodiments, the only regions of barcoding oligonucleotides fully complementary to the amplification product of the sequencing oligonucleotides are the primer regions.

### Methods

The invention features methods to generate amplicons using the oligonucleotide pairs of the invention as primers in one or more nucleic acid amplification reactions (e.g., PCR or RT-PCR), wherein the generated amplicons include a target nucleic acid sequence, an in-line barcode sequence and a pair of unique barcode sequences. The invention also features methods to sequence the generated amplicons described herein, wherein the sequences of the target nucleic acid sequence, in-line barcode sequence, and unique barcode sequences are determined to associate the target nucleic acid to a nucleic acid sample corresponding to the in-line barcode sequence and unique barcode sequences.

### Methods of Generating a Library

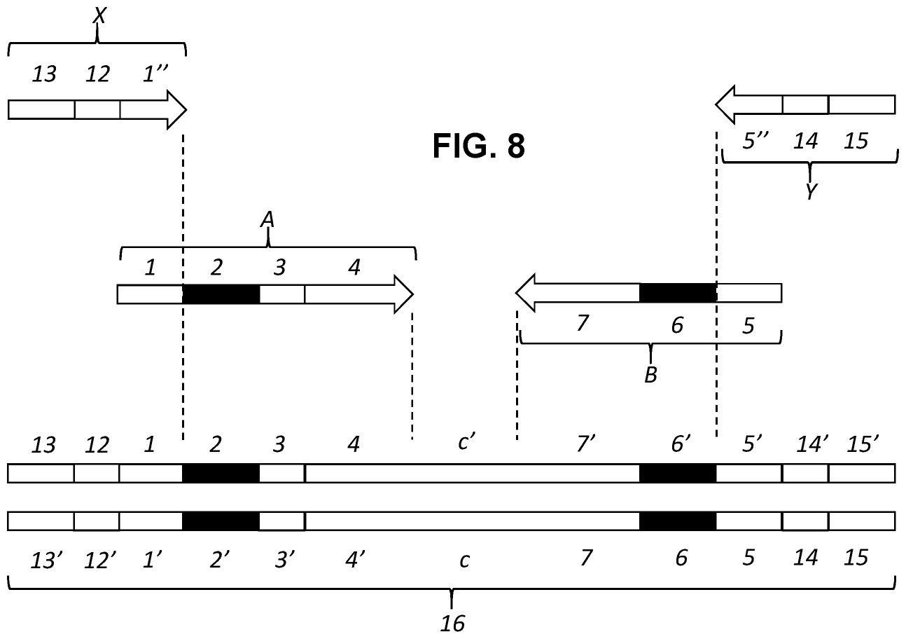

The invention further provides a method for the generation of a nucleic acid library of amplicons. As depicted in FIGS. 2-7, the amplicons in the nucleic acid library are generated by nucleic acid amplification reactions using the pair of sequencing oligonucleotides and pair of barcoding oligonucleotides. As depicted in FIG. 8, amplicons include a nucleic acid sequence having the first region for attachment to a solid substrate, the first unique barcode sequence, the first barcode primer region, the first sequencing primer region, the first in-line barcode region, the first target-specific binding region, the sequencing assay region, the complementary sequence of the second target-specific binding region, the complementary sequence of the second barcode primer region, the complementary sequence of the second unique barcode sequence, and the complementary sequence of the second region for attachment to a solid substrate, and the complement sequence thereof. If generated from more than one pair of sequencing oligonucleotides and/or more than one pair of barcoding oligonucleotides, amplicons within an amplified nucleic acid library may differ by their first in-line barcode sequences, their first unique barcode sequences, and their second unique barcode sequences. Typically, a different pair of sequencing oligonucleotides and/or a different pair of barcoding oligonucleotides will be used for the amplification of each nucleic acid sample to be pooled in a generated nucleic acid library, allowing for the different amplicons generated from different nucleic acid samples within a single nucleic acid library to be identified by their in-line barcode sequence and unique barcode sequences. For example, a nucleic acid library generated using a four pairs of sequencing oligonucleotides having four different first sequencing oligonucleotides with four different in-line barcodes and 384 pairs of barcoding oligonucleotides having 384 different first barcoding oligonucleotides and 384 different second barcoding oligonucleotides containing 384 different first and second unique barcode sequences, respectively, be used for 4 (different pairs of sequencing oligonucleotides)×384 (different pairs of barcoding oligonucleotides)=1536 different amplicons and complement sequences. In this manner, a kit containing plurality of pairs of sequencing and barcoding oligonucleotides can be used in a combinatorial manner to generate a nucleic acid library containing amplicons and complement sequences that are amplified from and that corresponding to at least 2, 3, 4, 5, 6, 7, 8, 9, 10, 16, 20, 25, 30, 40, 60, 70, 80, 90, 100, 200, 300, 400, 500, 1000, 2000, 3000, 5000, 10000, 20000, 30000, 50000, 100000 or more different nucleic acid samples. It will be understood that the unique barcode sequences will be employed more than once in sequencing, but only once in conjunction with a particular in-line barcoding region.

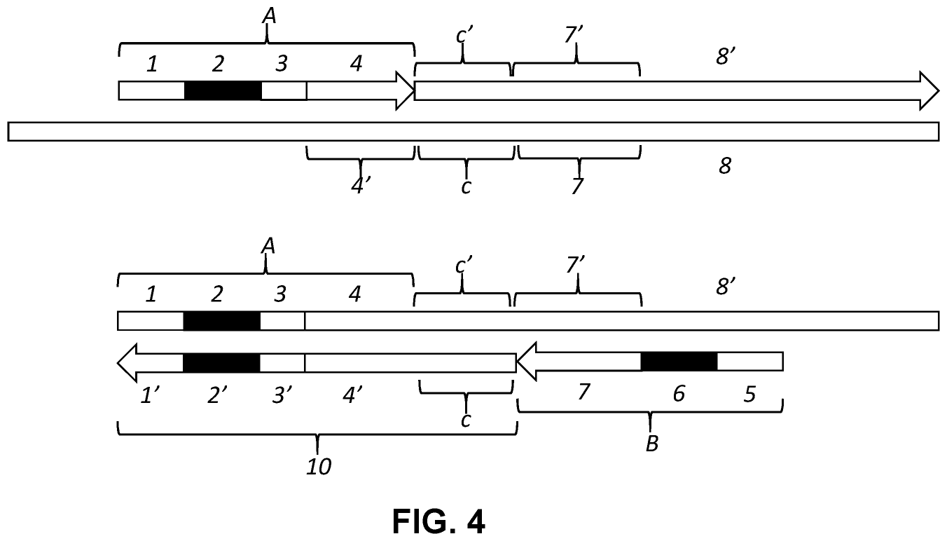

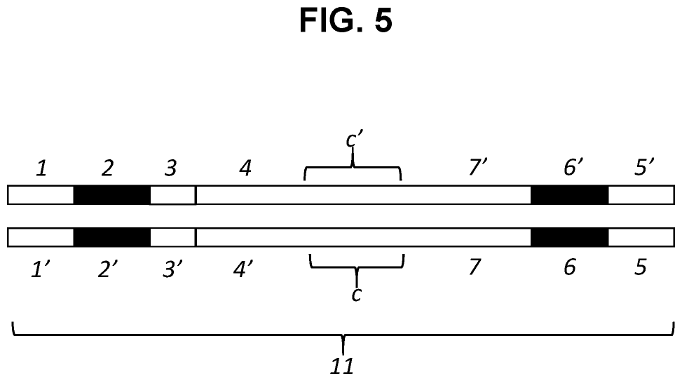

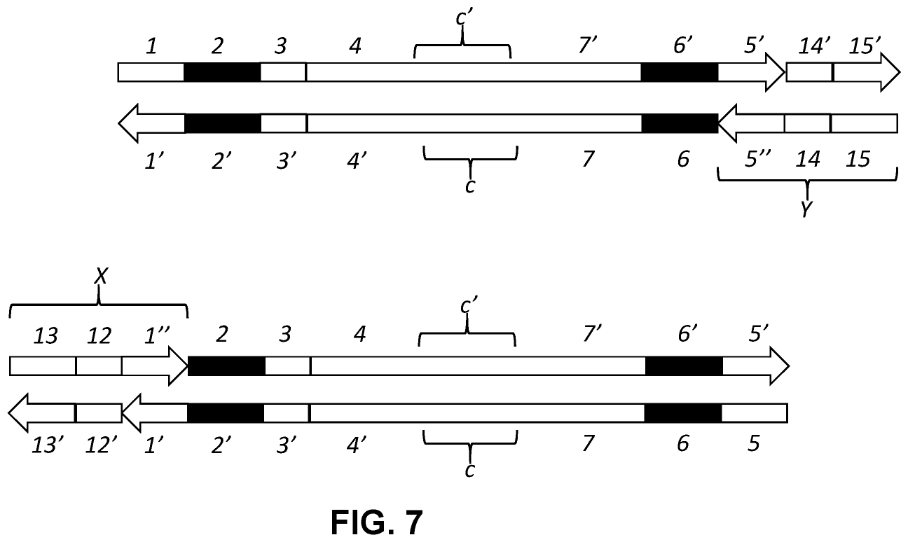

In certain embodiments, the one or more pairs of sequencing oligonucleotides and one or more pairs of barcoding oligonucleotides may be added simultaneously as primers in a single nucleic acid amplification reaction. In other embodiments, the pairs of sequencing oligonucleotides are first added as primers in a first amplification reaction, where, as depicted in FIG. 2, the first sequencing oligonucleotide hybridizes to a target nucleic acid via its first target-specific binding region. As depicted in FIG. 3, the first sequencing oligonucleotide will then act as a primer, allowing polymerase extension through the target nucleic acid and past the homologous region of the second target-specific binding region of the second sequencing oligonucleotide. This polymerase extension product can then allow hybridization of the second sequencing oligonucleotide via the second target-specific binding region, and, as depicted in FIG. 4, act as a primer in allowing another polymerase extension up to the complement sequence of the first barcode primer region. Multiple cycles of a nucleic acid amplification reaction using only a pair of sequencing oligonucleotides and a target nucleic acid as template generates multiple copies of an intermediate amplicon and its complement sequence, as depicted in FIG. 5. A pair of barcoding oligonucleotides can then be added in a second round of nucleic acid amplification reactions using the intermediate amplicons as templates. As depicted in FIG. 6, the first and second barcoding oligonucleotides hybridize to the intermediate amplicon and its complement sequence via the first and second primer regions, homologous to the first and second barcode primer regions, respectively. The pair of barcoding oligonucleotides then act as primers for polymerase extension (FIG. 7), the products of which can further bind a first or second barcoding oligonucleotide which act as primers for polymerase extension in subsequent cycles of nucleic acid amplification reaction to generate amplicons. The resulting amplicons include a first region for attachment to a solid substrate (13); a first unique barcode sequence (12); a first barcode primer region (1); a first sequencing primer region (2); a first in-line barcode region (3); a first target-specific binding region (4); a complement sequence (c′) of the sequencing assay region; a complement sequence (7′) of the second target-specific binding region; a complement sequence (6′) of the second sequencing primer region; a complement sequence (5′) of the second barcode primer region; a complement sequence (14′) of the second unique barcode sequence; and a complement sequence (15′) of the second region for attachment to a solid substrate; and their complement sequences, as depicted in FIG. 8.

In yet other embodiments, the pair of sequencing oligonucleotides may not contain a first and second target-specific binding region. Instead, the method would include two steps. In the first step, the in-line barcode region(s) may be added to the target nucleic acid via ligation of a pair of sequencing oligonucleotides that do not contain a first and second target-specific binding regions to produce intermediate amplicons. In the second step, the intermediate amplicons may be amplified using the pair of barcoding oligonucleotides, as described herein.

Nucleic acid amplification reactions described herein may involve at least 1, 2, 3, 4, 5, 6, 7, 8, 9, 10, 11, 12, 13, 14, 15, 16, 17, 18, 19, 20, 25, 30, or more cycles of amplification.

### Methods for the Sequencing of Amplicons

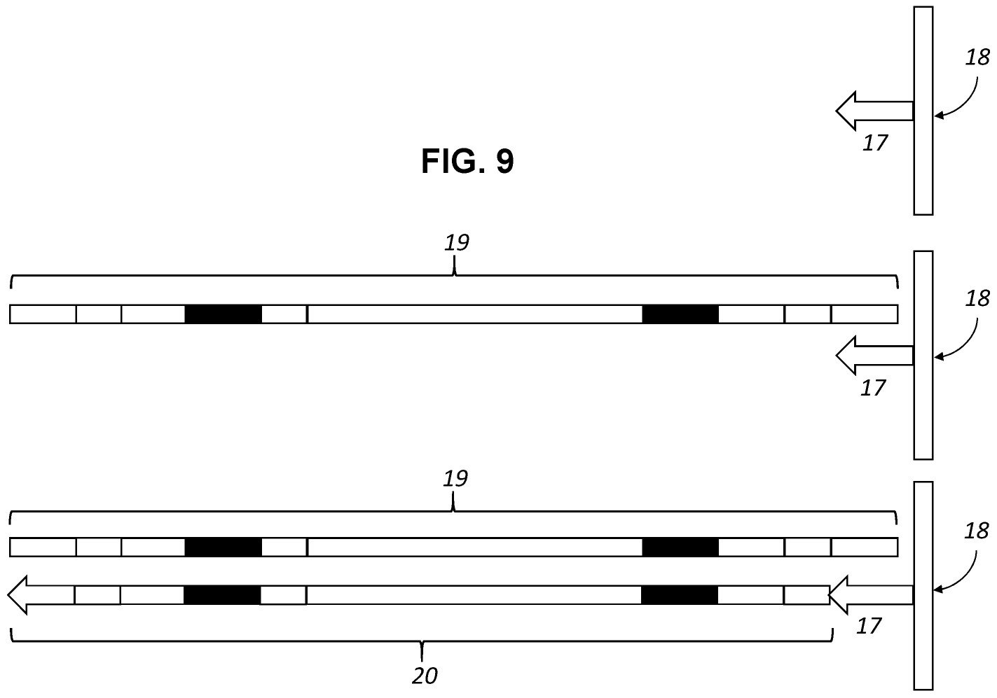

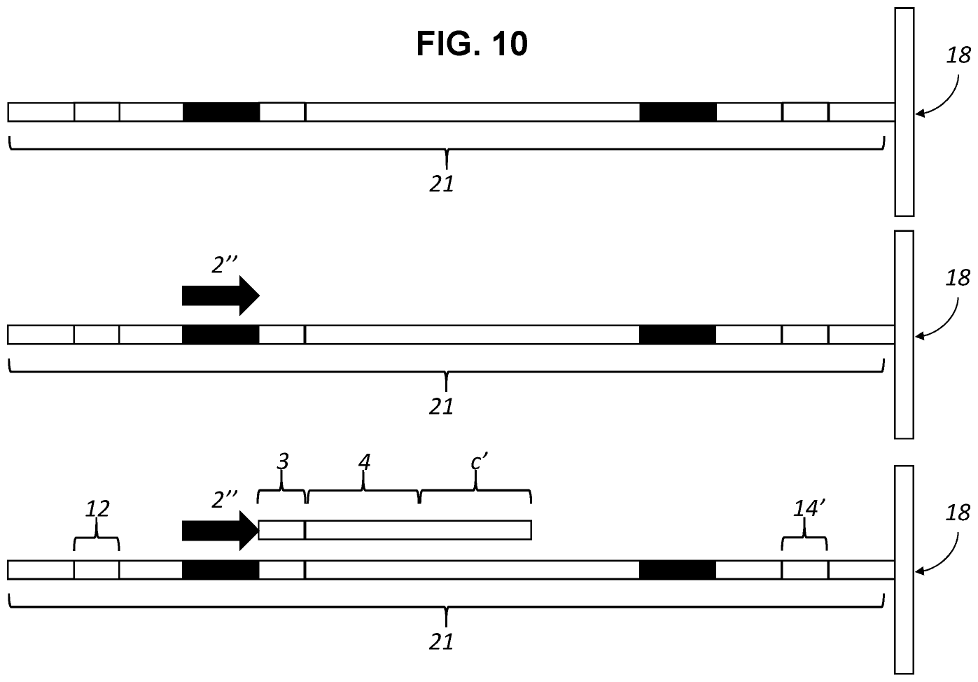

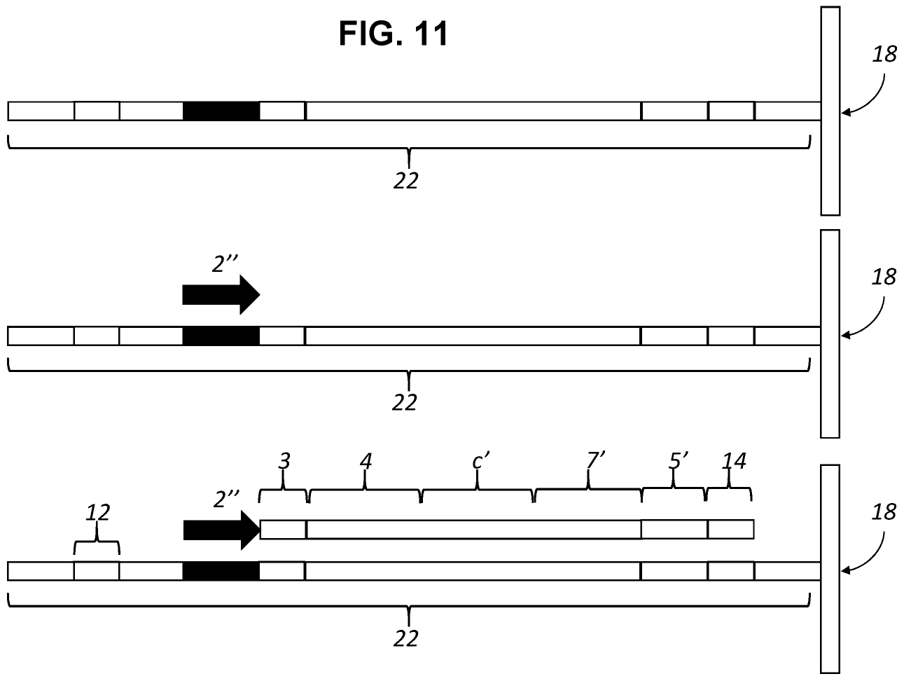

The invention further provides a method for the sequencing of a nucleic acid library of amplicons. As depicted in FIGS. 9-11, sequencing can be performed on a nucleic acid library of amplicons generated using the sequencing oligonucleotides and barcoding oligonucleotides of the invention. As depicted in FIG. 9, a portion of the amplicons and their complementary sequences are first hybridized to a solid substrate, and a covalently bound complement strand of nucleic acid is generated. As depicted in FIGS. 10 and 11, the first in-line barcode region, the first target specific binding region, and the sequence of the sequencing assay region are determined through sequencing-by-synthesis using a sequencing primer homologous to the first sequencing primer region, and the first and second unique barcode sequences are also sequenced, e.g., by separate index runs. This allows the amplicon, and the nucleic acid sample from which it is amplified, to be identified via the target nucleic acid sequence, the in-line barcode region, and the first and second unique barcoding sequences.

In some embodiments, the amplicons and their complement sequences are hybridized via their first or second regions for attachment to a solid substrate to a complementary primer region covalently bound to a solid surface (FIG. 9). In some embodiments, the covalently bound complement of the hybridized amplicon or complement sequence is generated through polymerase extension using the covalently bound primer region as a primer (FIG. 9).

In certain embodiments, as depicted in FIG. 10, the first and second unique barcode sequences are sequenced by index reads after the in-line barcode region is sequenced using sequence-by-synthesis. In other embodiments, as depicted in FIG. 11, the first unique barcode sequence is sequenced by index read, and the second unique barcode sequence is sequenced as part of the sequence-by-synthesis used to sequence the in-line barcode region.

In some embodiments, sequencing-by-ligation may be used to determine the sequences of the sequencing assay region, the first and second in-line barcode regions, and/or the first and second unique barcode sequences.

The sequencing may, for example, be performed on an NGS platform, though other methods of nucleic acid sequencing may be used. At least 1, 5, 10, 15, 20, 30, 40, 50, 75, 100, 200, 300, 400, 500, 1000, 2000, 3000, 5000, 7500, 10000, 50000, 100000, 500000, 750000 or more amplicons can be sequenced simultaneously. At least 1 million, 2 million, 3 million, 5 million, 10 million, 20 million, 30 million, million, 100 million, 200 million, 300 million, 500 million, 750 million, 1 billion, 2 billion, 3 billion, 4 billion, 5 billion, 6 billion, 7 billion, 8 billion, 9 billion, 10 billion, 11 billion, 12, billion, 13 billion, 14 billion, 15 billion, or more amplicons may be sequenced simultaneously.

### EXAMPLES

The invention will now be described by the following non-limiting examples.

### Example 1

Two-step amplicon library preparation procedure:
Materials:

### MagMAX Viral Pathogen kit (Thermo)

Heat-inactivated SARS-CoV-2 (ATCC)

TaqPath Master Mix (Thermo)

Pairs of sequencing oligonucleotides:
1. N1 (SEQ ID 1 and SEQ ID 2)
2. N2 (SEQ ID 3 and SEQ ID 4)
3. RP (SEQ ID 5 and SEQ ID 6)

Pairs of barcoding oligonucleotides (Illumina; UD Indexes Plate B/Set2: UDP0169-UDP0192)

MAGwise paramagnetic beads (seqWell)

SEQ ID 1:
TCGTCGGCAGCGTCAGATGTGTATAAGAGAC
AGCACTCACCGGACCCCAAAATCAGCGAAAT
SEQ ID 2:
GTCTCGTGGGCTCGGAGATGTGTATAAGAGA
CAGATAGAGAGTCTGGTTACTGCCAGTTGAA
TCTG
SEQ ID 3:
TCGTCGGCAGCGTCAGATGTGTATAAGAGAC
AGACCTTATGTTTACAAACATTGGCCGCAAA
SEQ ID 4:
GTCTCGTGGGCTCGGAGATGTGTATAAGAGA
CAGCCTCCATAGCGCGACATTCCGAAGAA
SEQ ID 5:
TCGTCGGCAGCGTCAGATGTGTATAAGAGAC
AGGATAGATCCAGATTTGGACCTGCGAGCG
SEQ ID 6:
GTCTCGTGGGCTCGGAGATGTGTATAAGAGA
CAGATGTATCAGATAGCAACAACTGAATAGC
CAAGGT

### Procedure:

1. Isolated total nucleic acid from human saliva with the MagMAX Viral Pathogen kit according to the manufacturer's instructions.
2. Prepared seven two-fold serial dilutions of heat-inactivated SARS-CoV-2 virus in 10 mM Tris-HCl, pH 8, starting from 1000 copies per reaction down to 16 copies per reaction.
3. Set up triplicate RT/PCR reactions (n=21) for each dilution of SARS-CoV-2,* by combining the following components in three 8-strip PCR tubes:

| Component | Volume (μL) |
|---|---|
| TaqPath Master Mix | 5 |
| Human nucleic acid | 5 |
| 10 μM N1/N2/RP Sequencing | |
| Oligonucleotides | 1 |
| SARS-CoV-2 dilution | 2 |
| 10 mM Tris-HCl, pH 8 | 7 |
| Total reaction volume | 20 |

* - Negative controls (n = 3) were also prepared that received all the reaction components listed above except SARS-CoV-2.

4. Transferred the capped 8-strip PCR tubes containing the reactions to a thermal cycler and ran the following RT-PCR thermal cycling program:

### RT-PCR Program (40 Cycles):

| Step | Temperature | Duration | |
|---|---|---|---|
| 1 | 25° C. | 2 | min |
| 2 | 53° C. | 10 | min |
| 3 | 95° C. | 2 | min |
| 4 | 95° C. | 3 | sec |
| 5 | 60° C. | 30 | sec |

| | | |
|---|---|---|
| 6 | (Return to step 3, 39X) | — |
| 7 | 4° C. | hold |

5. After completion of the RT-PCR thermal cycling program, set up barcode amplification reactions (n=24) by combining the following components in three new 8-strip PCR tubes:

| Component | Volume (μL) | |
|---|---|---|
| TaqPath Master Mix | 5 | (pre-heat |
| barcoding oligonucleotides (UDI) | 2.5 | inactivated)* |
| Aliquot from RT-PCR amplification | 1 | |
| 10 mM Tris-HCl, pH 8 | 11.5 | |
| Total reaction volume | 20 | |

*Pre-incubating the TaqPath Master Mix component at 95° C. for 5 minutes served to inactivate uracil DNA glycosylase before aliquots of uracil-containing RT-PCR amplification products were added to each barcode amplification reaction.

6. Transferred the capped 8-strip PCR tubes containing the reactions to a thermal cycler and ran the following barcode amplification thermal cycling program:

Barcode amplification program (10 cycles):

| Step | Temperature | Duration | |
|---|---|---|---|
| 1 | 95° C. | 2 | min |
| 2 | 95° C. | 3 | sec |
| 3 | 60° C. | 30 | sec |

| | | |
|---|---|---|
| 4 | (Return to step 2, 9X) | — |
| 5 | 4° C. | hold |

7. After completion of the barcode amplification thermal cycling program, the reaction products were pooled in a 1.5 mL tube that was preloaded with 75 mM EDTA to inhibit any residual DNA polymerase activity that might have been present.
8. MAGwise beads were mixed with the pooled barcoded amplification products in 1.5:1 volumetric ratio and allowed to bind for 5 minutes at room temperature.
9. The tube was transferred to a magnetic tube holder and after the bead pellet formed, the supernatant fluid was removed and discarded.
10. The bead pellet was washed two times with 500 μl of 80% ethanol. After each ethanol wash, the supernatant fluid was removed and discarded.
11. The tube was removed from the magnetic tube holder and the bead pellet was resuspended in 50 μl of 10 mM Tris-HCl, pH 8.
12. After eluting the purified DNA for 5 minutes at room temperature, the tube was returned to the magnetic tube holder.
13. After the bead pellet formed, the eluate (containing the purified pooled library) was transferred to a new 1.5 mL tube.
14. Aliquots of the purified library were analyzed by gel electrophoresis and quantified by qPCR.
15. The quantified library was diluted to 4 nM, denatured with 0.2 N sodium hydroxide, and loaded on to an Illumina MiSeq Micro v2 cartridge according to the manufacturer's instructions. The MiSeq sequencing configuration was set-up for dual-indexed sequencing, as follows:
Read 1—(60 bases) The sequence identifier (in-line) and the target DNA were read.
Read 2—(10 bases) The i7 barcode index was read.
Read 3—(10 bases) The i5 barcode index was read.

### Results and Data Analysis:

After demultiplexing the sequencing results from the MiSeq run, the number of reads with exact matches to the first 9 bases, corresponding to the in-line barcode region and the 50 bases of the N1, N2 and RP amplicons were counted (see below) for each sample:

N1_Read_1
(SEQ ID NO. 7)
CACTCACCGGACCCCAAAATCAGCGAAATGCA
CCCCGCATTACGTTTGGTGGACCCTCA
N2_Read_1
(SEQ ID NO. 8)
ACCTTATGTTTACAAACATTGGCCGCAAATTG
CACAATTTGCCCCCAGCGCTTCAGCGT
RP_Read_1
(SEQ ID NO. 9)
GATAGATCCAGATTTGGACCTGCGAGCGGGTT
CTGACCTGAAGGCTCTGCGCGGACTTG

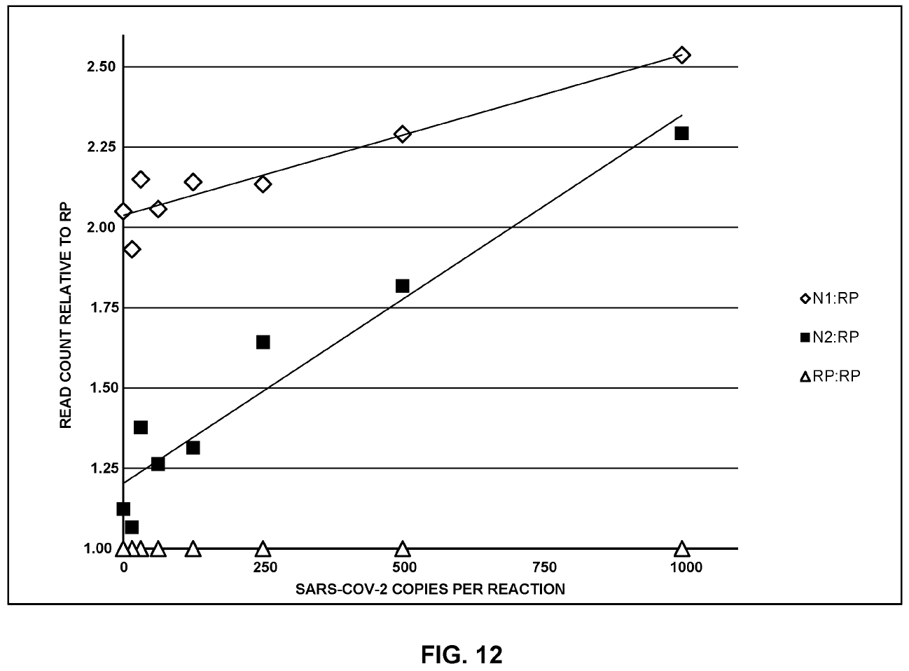

The results for Example 1 are shown in FIG. 12.

### Example 2

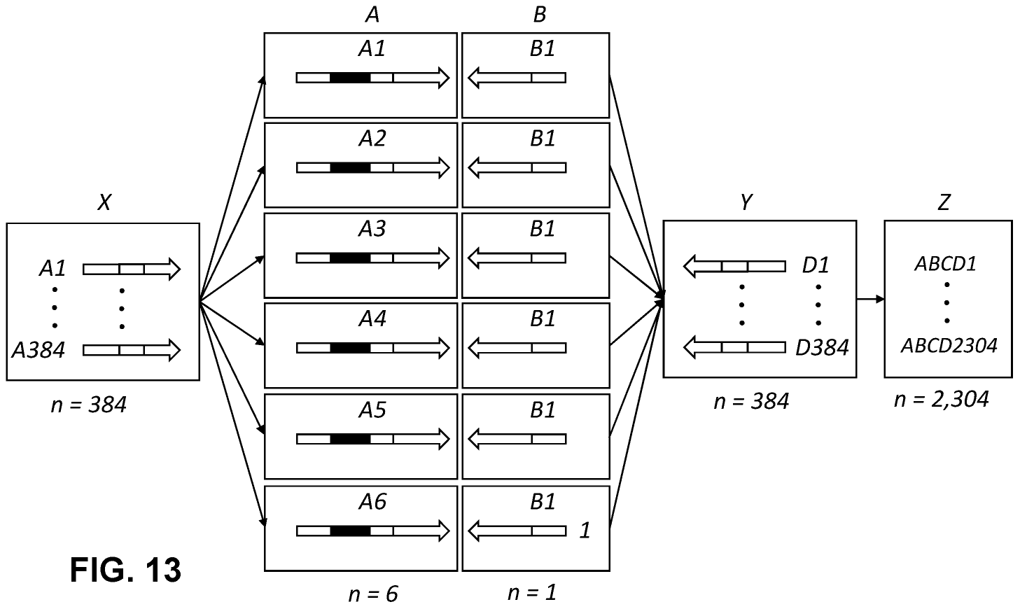

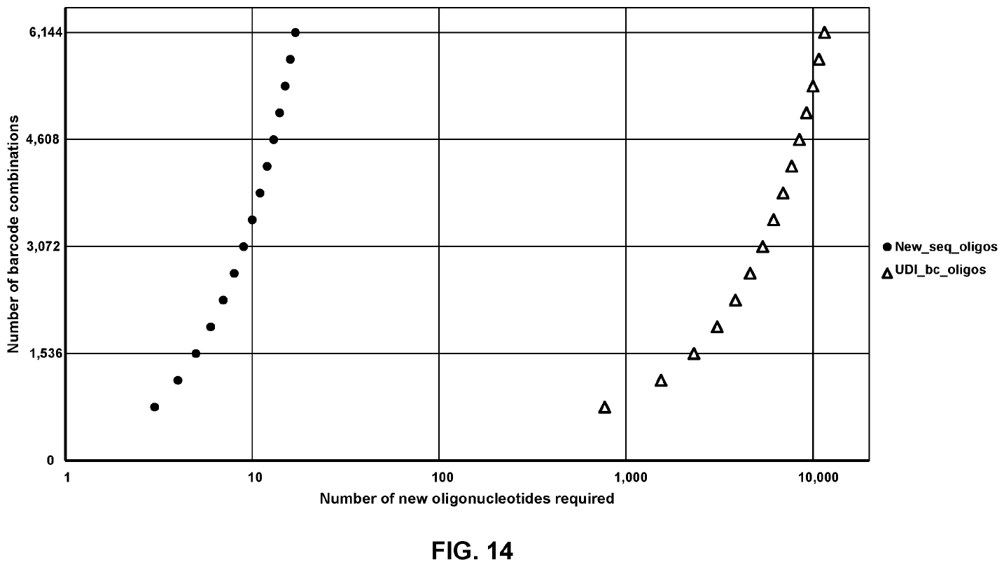

The number of barcode combinations can be increased by using sequencing oligonucleotides with in-line barcode regions in conjunction with a set of barcoding oligonucleotides. A set of 384 barcoding oligonucleotides combinations can be expanded to 768 barcode combinations by only adding two pairs of oligonucleotides which include three new oligonucleotide sequences: two first sequencing oligonucleotides with different in-line barcode sequences and a second sequencing oligonucleotide. See the chart in FIGS. 13 and 14.

What is claimed is:

1. A pair of sequencing oligonucleotides comprising:
(a) a first oligonucleotide comprising from 5′ to 3′ a first barcode primer region, a first sequencing primer region, a first in-line barcode region, and a first target-specific binding region complementary to a first sequence in a target nucleic acid; and
(b) a second oligonucleotide comprising from 5′ to 3′ a second barcode primer region and a second target-specific binding region homologous to a second sequence in the target nucleic acid, wherein the first and second target-specific binding regions flank a sequencing assay region in the target nucleic acid that can be amplified using the pair.

2. The pair of claim 1, wherein the second oligonucleotide further comprises a second sequencing primer region between the second barcode primer region and the second target-specific binding region.

3. The pair of claim 1, wherein the second oligonucleotide further comprises a second in-line barcode region between the second barcode primer region and the second target-specific binding region.

4. The pair of claim 1, wherein the first and second oligonucleotides comprise RNA.

5. The pair of claim 1, wherein the first and second oligonucleotides comprise DNA.

6. A kit comprising a plurality of pairs of claim 1, wherein the sequence of the first in-line barcode region for each first oligonucleotide is different.

7. A kit comprising (a) a pair of sequencing oligonucleotides of claim 1 and (b) a pair of barcoding oligonucleotides comprising:
(i) a first barcoding oligonucleotide comprising from 5′ to 3′ a first region for attachment to a solid substrate, a first unique barcode sequence, and a first primer region homologous to the first barcode primer region; and
(ii) a second barcoding oligonucleotide comprising a second region for attachment to a solid substrate, a second unique barcode sequence, and a second primer region homologous to the second barcode primer region.

8. The kit of claim 7, further comprising a plurality of pairs of sequencing oligonucleotides, wherein the sequence of the first in-line barcode region for each first oligonucleotide is different, and/or a plurality of pairs of barcoding oligonucleotides, wherein the sequence of the first unique barcode sequence for each first barcoding oligonucleotide is different.

9. The kit of 8, wherein the sequence of the second unique barcode sequence for each second barcoding oligonucleotide is different.

10. A method of generating a library from a nucleic acid sample comprising amplifying the nucleic acid sample using the kit of claim 5 to produce amplicons, wherein the amplicons comprise a nucleic acid sequence comprising the first region for attachment to a solid substrate, the first unique barcode sequence, the first barcode primer region, the first sequencing primer region, the first in-line barcode region, the first target-specific binding region, the sequencing assay region, the complement sequence of the second target-specific binding region, the complement sequence of the second barcode primer region, the complement sequence of the second unique barcode sequence, and the complement sequence of the second region for attachment to a solid substrate and the complement thereof, thereby generating the library.

11. The method of claim 10, wherein the nucleic acid sample is amplified using the pair of sequencing oligonucleotides and the pair of barcoding oligonucleotides in a single amplification step to produce the amplicons.

12. The method of claim 10, wherein the nucleic acid sample is sequentially amplified by
(a) amplifying the nucleic acid sample using the pair of sequencing oligonucleotides to produce an intermediate amplicon comprising a nucleic acid sequence comprising the first barcode primer region, the first sequencing primer region, the first in-line barcode region, the first target-specific binding region, the sequencing assay region, the complement sequence of the second target-specific binding region, and the complement sequence of the second barcode primer region and the complement thereof; and
(b) amplifying the intermediate amplicon and its complement using the pair of barcoding oligonucleotides to produce the amplicons.

13. A method of sequencing a target nucleic acid sequence in a nucleic acid sample comprising the steps of
(a) providing amplicons comprising a nucleic acid sequence comprising the first region for attachment to a solid substrate, the first unique barcode sequence, the first barcode primer region, the first sequencing primer region, the first in-line barcode region, the first target-specific binding region, the sequencing assay region, the complement sequence of the second target-specific binding region, the complement sequence of the second barcode primer region, the complement sequence of the second unique barcode sequence, and the complement sequence of the second region for attachment to a solid substrate and the complement thereof;
(b) hybridizing at least a portion of the amplicons to a solid substrate and creating a covalently bound complement thereof;
(c) sequencing the first in-line barcode region, the first target specific binding region, and the sequencing assay region through sequencing-by-synthesis using a sequencing primer homologous to the first sequencing primer region; and
(d) sequencing the first and second unique barcode sequences of the amplicon.

14. The method of claim 13, wherein step (b) comprises hybridizing the amplicons to immobilized primers covalently attached to the solid substrate, wherein the immobilized primers are homologous to the first or second region for attachment.

15. The method of claim 14, wherein the immobilized primer is used to generate a complement of the hybridized amplicon through polymerase extension.

16. The method of claim 13, wherein the first and second unique barcode sequences are sequenced by index reads.

17. The method of claim 13, wherein the second unique barcode sequence is sequenced in-line after step (c).
